(19) United States
(12) Patent Application Publication
(10) Pub. No.: US 2026/0244263 A1
(43) Pub. Date: Aug. 20, 2026

(54) CONTROL DEVICE, CONTROL METHOD, AND CONTROL PROGRAM

(71) Applicant: SONY SEMICONDUCTOR SOLUTIONS CORPORATION, KANAGAWA (JP)

(72) Inventors: HIROMASA DOI, KANAGAWA (JP); HIROYUKI KASAHARA, KANAGAWA (JP); KOHEI SHODA, KANAGAWA (JP)

(21) Appl. No.: 19/472,377

(22) PCT Filed: Mar. 18, 2024
(86) PCT No.: PCT/JP2024/010501

§ 371 Date: Oct. 3, 2025

(30) Foreign Application Priority Data
Apr. 13, 2023 (JP) 2023-065862

(51) Int. Cl. G06F 3/01; G06T 7/73

(52) U.S. Cl. CPC G06F 3/012; G06T 7/73; G06T 2207/30201

(57) ABSTRACT

To enable the posture of an avatar to be controlled with high accuracy. A control device includes an acquisition unit that acquires position information on fingers and elbows related to positions of fingers and elbows of a user, a calculation unit that calculates elbow joint orientation information related to orientations of joints of the elbows, based on the position information on the fingers and the elbows acquired by the acquisition unit, and a model control unit that controls the posture of an avatar of the user, based on the elbow joint orientation information calculated by the calculation unit.

### TECHNICAL FIELD

The present invention relates to a control device, a control method, and a control program.

### BACKGROUND ART

In recent years, a motion capture technique for acquiring a user body motion information has been actively developed. The acquired body motion information is used for applications such as virtual reality (VR) and augmented reality (AR) (for example, see PTL 1).

### CITATION LIST

### Patent Literature

PTL 1: WO 2019/203188

### SUMMARY

### Technical Problem

However, in the above-described technique, a posture of an avatar may not be controlled with high accuracy. For example, in the above-described technique, since the rotation angle of a joint or the like is not taken into consideration, the posture of an avatar may not be controlled with high accuracy. An aspect of the present disclosure enables a posture of an avatar to be controlled with high accuracy.

### Solution to Problem

A control device according to an aspect of the present disclosure includes an acquisition unit configured to acquire position information on fingers and elbows related to positions of fingers and elbows of a user, a calculation unit configured to calculate elbow joint orientation information related to orientations of joints of the elbows, based on the position information on the fingers and the elbows acquired by the acquisition unit, and a control unit configured to control a posture of an avatar of the user, based on the elbow joint orientation information calculated by the calculation unit.

A control method according to an aspect of the present disclosure is a control method executed by a control device, the control method including an acquisition step of acquiring position information on fingers and elbows related to positions of fingers and elbows of a user, a calculation step of calculating elbow joint orientation information related to orientations of joints of the elbows, based on the position information on the fingers and the elbows acquired in the acquisition step, and a control step of controlling a posture of an avatar of the user, based on the elbow joint orientation information calculated in the calculation step.

A control program according to an aspect of the present disclosure causes a computer mounted on a control device to execute acquisition processing of acquiring position information on fingers and elbows related to positions of fingers and elbows of a user, calculation processing of calculating elbow joint orientation information related to orientations of joints of the elbows, based on the position information on the fingers and the elbows acquired by the acquisition processing, and control processing of controlling a posture of an avatar of the user, based on the elbow joint orientation information calculated by the calculation processing.

### BRIEF DESCRIPTION OF DRAWINGS

FIG. 1 is a diagram depicting an example of a schematic configuration of a control device.

FIG. 2 is a diagram depicting an example of an outline of processing by the control device.

FIG. 3 is a diagram depicting an example of an outline of processing by the control device.

FIG. 4 is a diagram depicting an example of an outline of processing by the control device.

FIG. 5 is diagrams for explaining an example of the control device.

FIG. 6 is a diagram for explaining an example of the control device.

FIG. 7 is a diagram for explaining an example of the control device.

FIG. 8 is diagrams for explaining an example of the control device.

FIG. 9 is a diagram for explaining an example of the control device.

FIG. 10 is a diagram for explaining an example of the control device.

FIG. 11 is a diagram depicting an example of a flow of processing by the control device.

FIG. 12 is a diagram depicting an example of a hardware configuration of a device.

FIG. 13 is a diagram for explaining an example of a reference technique.

FIG. 14 is diagrams for explaining an example of the reference technique.

### DESCRIPTION OF EMBODIMENTS

Embodiments of the present disclosure will be explained below in detail with reference to the drawings. In the following embodiments, the same elements are denoted by the same reference numerals, and redundant descriptions may be omitted.

The present disclosure will be described in the following item order.
0. Introduction
1. Embodiments
2. Example of Hardware Configuration
3. Example of Effect

### 0. Introduction

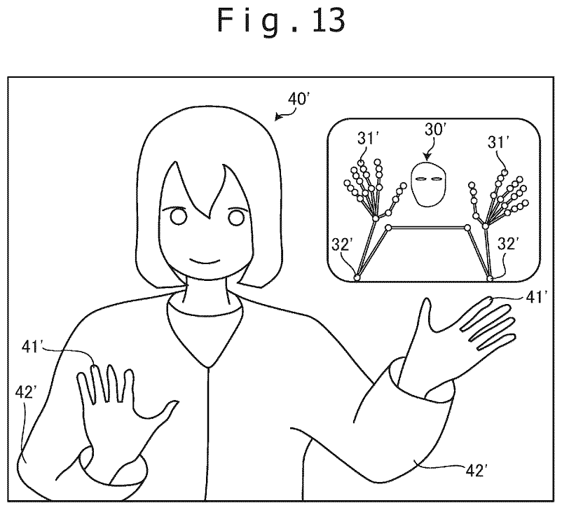

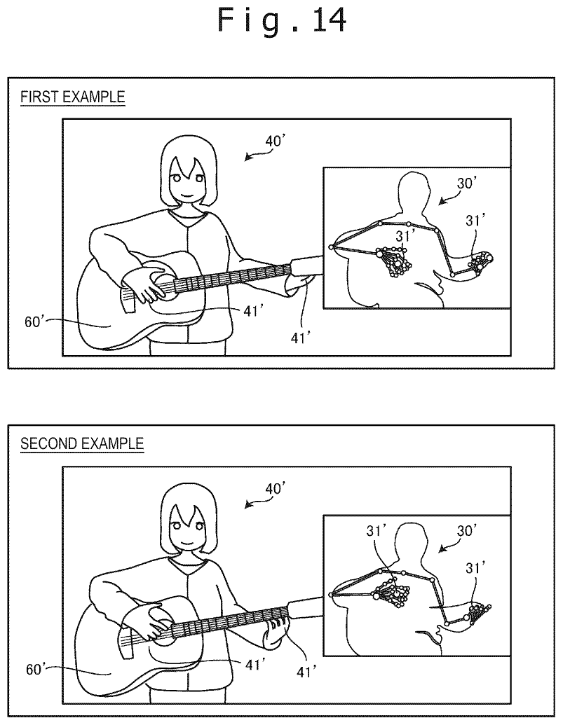

As described above, the technique as in PTL 1 may not be able to control a posture of an avatar with high accuracy. For example, in the above-described technique, since the rotation angle of a joint or the like is not taken into consideration, the posture of an avatar may not be controlled with high accuracy. A reference technique, which is an example of the above-described technique, will be explained below with reference to FIGS. 13 and 14. FIGS. 13 and 14 are diagrams for explaining examples of the reference technique.

First, as a premise, inverse kinematics (IK) processing will be explained. In an articulated object, an orientation of a joint, such as a rotation angle of a joint, changes in accordance with a position of the joint. The IK processing is, for example, processing of calculating the rotation angle of a joint when a position of the tip of an object is determined.

Next, an example of an issue of the reference technique will be explained with reference to FIG. 13. In the example depicted in FIG. 13, the reference technique does not perform IK processing of calculating the rotation angles of joints of elbows 32′ of a user 30′, and controls a posture of an avatar 40′ of the user 30′, based on a recognition result of imaging information on the user 30′, obtained by imaging the user 30′. In such a technique, for example, when the posture of the avatar 40′ is controlled by calculating the rotation angles of the base of the arms of the user 30′, positions of the elbows 42′ of the avatar 40′ are displaced, and particularly, positions of fingers 41′, which are tip positions of the arms, are largely displaced. In addition, when the user 30′ touches their face with their hand, or the user 30′ claps their hands, the positions of the fingers 41′ of the avatar 40′ may be particularly displaced, and the avatar 40′ may move in an unusual manner. As described above, in the reference technique, the posture of the avatar 40′ may not be controlled with high accuracy.

Next, another example of the issue of the reference technique will be explained with reference to FIG. 14. In the example depicted in FIG. 14, the reference technique causes a guitar 60′ to persistently follow a position and an orientation of a waist of a computer graphics (CG) model, such as a virtual reality model (VRM), which is the avatar 40′ of the user 30′. Thus, the reference technique is not necessarily recognizing the position and orientation of the guitar in the real world where the user 30′ is playing. Therefore, as depicted in the diagrams of a first example and a second example in FIG. 14, the reference technique may dispose the guitar 60′ at a position irrelevant to that in the real world even when the fingers 41′ of the avatar 40′ are controlled to be at correct positions corresponding to fingers 31′ of the user 30′ in the real world. As a result, even if the user 30′ is playing the guitar with a neck of the guitar being pressed in a normal manner in the real world, positions of the guitar 60′ and the fingers 31′ may be misaligned on the CG. For example, as depicted in the drawing of the first example of FIG. 14 and the drawing of the second example of FIG. 14, in the reference technique, due to erroneous recognition, control occurs that is not appropriate in terms of the skeletal structure, such as a neck of the guitar 60′ not being pressed by the finger 41′ of a left hand of the avatar 40′. As described above, in the reference technique, when the user 30′ is in a specific posture, such as a posture when the user 30′ is singing while playing the guitar, the posture of the avatar 40′ may not be controlled with high accuracy.

In contrast, according to the technique of the present disclosure, it is possible to control the posture of the avatar with high accuracy. A specific technique will be explained in embodiments described below.

### 1. Embodiments

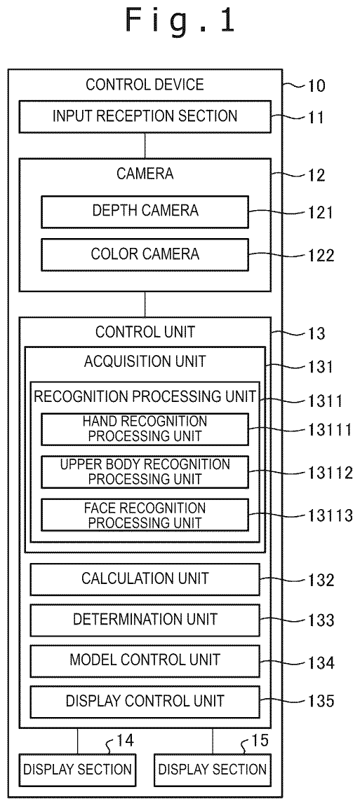

FIG. 1 is a diagram depicting an example of a schematic configuration of a control device. A control device 10 acquires position information on fingers and elbows, calculates elbow joint orientation information, based on the acquired position information on the fingers and elbows, and controls the posture of the avatar of the user, based on the calculated elbow joint orientation information. For example, the control device 10 controls the posture of the avatar, based on the calculated elbow joint orientation information, so that the fingers, which are tip portions of arms of the avatar of the user, are positioned at natural positions in accordance with orientations of joints of the elbows.

The position information on the fingers and the elbows refers to information related to positions of fingers and elbows of the user. The position information on the fingers and the elbows may include information related to positions of fingers of the avatar, which are calculated based on the information related to the positions of the fingers of the user, and information related to positions of elbows of the avatar, which are calculated based on the information related to the positions of the elbows of the user. The elbow joint orientation information refers to information related to orientations of joints of the elbows of the user. The elbow joint orientation information may include the information related to the positions of the elbows of the user, the information related to the positions of the elbows of the avatar, or the information related to the orientations of the joints of the elbows of the avatar, which is calculated based on the information related to the orientations of the joints of the elbows of the user.

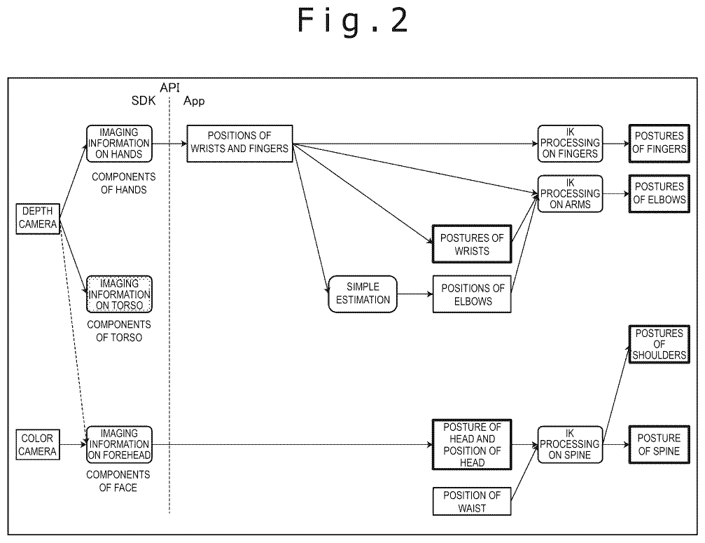

Examples of the control device 10 as described above include a smartphone and a personal computer. Before explaining a configuration of the control device 10, examples of an outline of processing by the control device 10 will be explained below with reference to FIGS. 2 to 4. FIGS. 2 to 4 are diagrams depicting examples of the outline of the processing by the control device. First, with reference to FIG. 2, an example of the outline of the processing by the control device 10 in a case where the control device 10 does not control the posture of the avatar, based on imaging information on a torso obtained by imaging a torso of the user, will be explained.

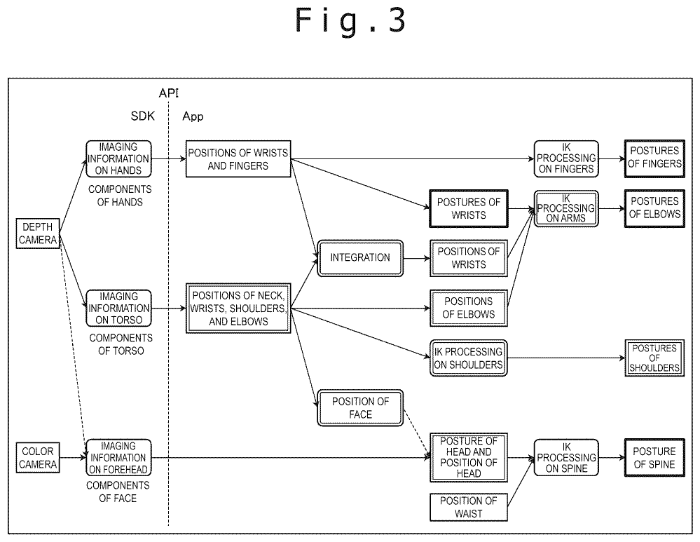

Hereinafter, in the examples depicted in FIGS. 2 and 3, a case where the control device 10 acquires position information related to a position of a body part of the avatar, based on position information related to a position of a body part of the user will be mainly explained. The following explanation will mainly focus on a case where the control device 10 calculates body part joint orientation information related to an orientation of a joint of the body part of the avatar, based on the acquired position information on the body part of the avatar, rather than on the acquired position information on the body part of the user. Further, the following explanation will mainly focus on a case where the control device 10 controls the posture of the avatar, based on the calculated body part joint orientation information related to the orientation of the joint of the body part of the avatar, rather than on body part joint orientation information related to an orientation of a joint of the body part of the user. However, the control device 10 may calculate the body part joint orientation information related to the orientation of the joint of the body part of the user, based on the acquired position information on the body part of the user, and may control the posture of the avatar, based on the calculated body part joint orientation information.

In the example depicted in FIG. 2, the control device 10 acquires imaging information on hands of the user and imaging information on the torso of the user (and imaging information on a face of the user) obtained by imaging by a depth camera. For example, the control device 10 acquires the imaging information on the hands, the imaging information on the torso, and the imaging information on the face of the user obtained by imaging by a depth camera in a software development kit (SDK) suitable for an augmented reality (AR) application. The control device 10 also acquires imaging information on the face of the user obtained by imaging by a color camera.

The imaging information on the hands includes imaging information on components of the hands such as imaging information on fingers. The imaging information on the torso includes imaging information on components of the torso such as imaging information on elbows. The imaging information on the face includes imaging information on components of the face. The programming language and the file format used internally for the imaging information, such as the imaging information on the hands, the imaging information on the torso, and the imaging information on the face may be freely chosen.

The control device 10 acquires position information on wrists and fingers related to positions of wrists and fingers of the user, based on the imaging information on the hands of the user. The control device 10 calculates position information on wrists and fingers of the avatar of the user corresponding to the position information on the wrists and the fingers of the user, based on the acquired position information on the wrists and the fingers of the user. For example, the control device 10 calculates the position information on the wrists and the fingers of the avatar corresponding to the position information on the wrists and the fingers of the user, using application software (App) connected to the SDK by an application programming interface (API).

The control device 10 performs IK processing or the like on fingers for calculating the rotation angles of joints of the fingers of the avatar, based on the calculated position information on the wrists and the fingers of the avatar, and calculates finger joint orientation information related to orientations of the joints of the fingers of the avatar. The control device 10 controls postures of the fingers of the avatar, based on the calculated finger joint orientation information.

The control device 10 controls postures of the wrists of the avatar corresponding to the wrists of the user, based on the calculated position information on the wrists and the fingers of the user or the avatar. The control device 10 may control the postures of the wrists of the avatar, based additionally on fist joint orientation information related to orientations of joints of fists of the user or the avatar. For example, the control device 10 controls the postures of the wrists of the avatar, based on the acquired position information on the wrists and the fingers of the avatar and the acquired fist joint orientation information on the avatar. The control device 10 may directly acquire the fist joint orientation information from input information, or may indirectly acquire the fist joint orientation information from calculation based on the imaging information on the user.

The control device 10 simply estimates the position information on the elbows of the avatar, based on the calculated position information on the wrists and the fingers of the user or the avatar. The control device 10 calculates arm joint orientation information related to orientations of joints of arms of the avatar, based on the acquired position information on the wrists and the fingers of the user or the avatar, posture information on the wrists of the avatar, and the simply-estimated position information on the elbows of the avatar. For example, the control device 10 performs IK processing or the like on arms for calculating the rotation angles of joints of the arms of the avatar, based on the position information on the wrists and the fingers of the user or the avatar, the posture information on the wrists of the avatar, and the position information on the elbows of the avatar, and calculates the arm joint orientation information on the avatar including elbow joint orientation information related to orientations of joints of the elbows of the avatar. The posture information refers to information related to a posture of a body part. The posture information may include information related to a joint of the body part or an orientation of the body part.

The control device 10 controls postures of the elbows of the avatar, based on the calculated arm joint orientation information on the avatar. For example, the control device 10 controls the postures of the elbows of the avatar, based on the elbow joint orientation information on the avatar included in the calculated arm joint orientation information on the avatar.

The control device 10 acquires posture information on a head and position information on a head of the user, based on the acquired imaging information on the face. Further, the control device 10 calculates posture information on a head and position information on a head of the avatar corresponding to the posture information on the head and position information on the head of the user. The control device 10 controls a posture of the head and a position of the head of the avatar, based on the calculated posture information and position information on the head of the avatar.

The control device 10 calculates spine joint orientation information related to an orientation of a joint of a spine of the avatar, based on the calculated posture information and position information on the head of the avatar and position information on a waist of the avatar. In this case, for example, the control device 10 first calculates, as the position information on the waist of the avatar, a position vertically below the head of the avatar by a predetermined distance, based on the position of the head of the avatar in a horizontal direction (X-axis direction) and a vertical direction (Z-axis direction), obtained after calibration has been performed. Subsequently, the control device 10 performs IK processing or the like on a spine for calculating the rotation angle of a joint of the spine of the avatar, based on the calculated posture information and position information on the head of the avatar and the calculated position information on the waist of the avatar, and calculates spine joint orientation information on the avatar.

The control device 10 controls postures of shoulders of the avatar, based on the calculated spine joint orientation information on the avatar. The control device 10 controls a posture of the spine of the avatar, based on the calculated spine joint orientation information on the avatar.

As described above, in the example depicted in FIG. 2, the control device 10 controls the postures of the fingers, the postures of the wrists, the postures of the elbows, the position of the head, the posture of the head, the postures of the shoulders, and the posture of the spine of the avatar, as depicted in the sections enclosed by bold lines.

Next, an example of the outline of the processing by the control device 10, in a case where the control device 10 controls the posture of the avatar, based on the imaging information on the torso obtained by imaging the torso of the user, will be described with reference to FIG. 3. Only the processing that is enclosed by double frame lines and different from the processing depicted in FIG. 2 will be explained below.

The control device 10 acquires position information on a neck, wrists, shoulders, and elbows of the user related to positions of the neck, wrists, shoulders, and elbows of the user, based on the acquired imaging information on the torso of the user. The control device 10 acquires position information on a neck, wrists, shoulders, and elbows of the avatar related to positions of the neck, wrists, shoulders, and elbows of the avatar, based on the position information on the neck, the wrists, the shoulders, and the elbows of the user. The control device 10 integrates the position information on the wrists and fingers of the avatar and the calculated position information on the neck, wrists, shoulders, and elbows of the avatar into position information on the neck, wrists, shoulders, fingers, and elbows of the avatar related to positions of the neck, wrists, shoulders, fingers, and elbows of the avatar. For example, the control device 10 integrates the position information on the wrists and fingers of the avatar and the position information on the neck, wrists, shoulders, and elbows of the avatar into the position information on the neck, wrists, shoulders, fingers, and elbows of the avatar.

The control device 10 calculates position information on the wrists of the avatar, based on the integrated position information on the neck, wrists, shoulders, fingers, and elbows of the avatar. The control device 10 estimates the position information on the elbows of the avatar, based on the position information on the neck, wrists, shoulders, and elbows of the avatar. The control device 10 calculates the arm joint orientation information related to the orientations of the joints of the arms of the avatar, based on the posture information on the wrists of the avatar, the calculated position information on the wrists of the avatar, and the estimated position information on the elbows of the avatar. For example, the control device 10 performs IK processing or the like on arms for calculating the rotation angles of joints of the arms of the avatar, based on the posture information on the wrists of the avatar, the calculated position information on the wrists of the avatar, and the estimated position information on the elbows of the avatar, and calculates the arm joint orientation information on the avatar including the elbow joint orientation information related to the orientations of the joints of the elbows of the avatar.

The control device 10 calculates shoulder joint orientation information on the avatar related to orientations of joints of the shoulders of the avatar, based on the calculated position information on the neck, wrists, shoulders, and elbows of the avatar. For example, the control device 10 performs IK processing or the like on shoulders for estimating rotation angles of the joints of the shoulders of the avatar in a direction (Y-axis direction) facing a screen displayed on the control device 10, based on the calculated position information on the neck, wrists, shoulders, and elbows of the avatar. Further, the control device 10 performs the IK processing on shoulders for estimating the rotation angles of the joints of the shoulders of the avatar in the vertical direction (up-down direction, Z-axis direction), based on the calculated position information on the neck, wrists, shoulders, and elbows of the avatar.

The control device 10 controls the postures of the shoulders of the avatar, based on the calculated shoulder joint orientation information on the avatar. For example, the control device 10 controls the postures of the shoulders of the avatar, based on the estimated rotation angles of the joints of the shoulders of the avatar with respect to the Y-axis direction, and the estimated rotation angles of the joints of the shoulders of the avatar with respect to the Z-axis direction.

When the imaging information on the face of the user is lost, the control device 10 estimates position information on a face of the avatar related to a position of the face of the avatar, based on the position information on the neck, wrists, shoulders, and elbows of the avatar. When the imaging information on the face of the user is lost, the control device 10 may further estimate posture information on the face of the avatar related to a posture of the face of the avatar, based on the position information on the neck, wrists, shoulders, and elbows of the avatar. For example, when the imaging information on the face of the user is lost, the control device 10 estimates the position information on the face of the avatar and the posture information on the face of the avatar, based on the position information on the neck, wrists, shoulders, and elbows of the avatar.

When the imaging information on the face of the user is lost, the control device 10 calculates the posture information on the head and the position information on the head of the avatar, based on the estimated position information on the face of the avatar. The control device 10 controls the posture of the head and the position of the head of the avatar, based on the calculated posture information and position information on the head of the avatar.

As described above, in the example depicted in FIG. 3 as well, the control device 10 controls the postures of the fingers, the postures of the wrists, the postures of the elbows, the position of the head, the posture of the head, the postures of the shoulders, and the posture of the spine of the avatar as depicted in sections enclosed by bold lines in FIG. 3. However, the example depicted in FIG. 3 is different from the example depicted in FIG. 2 in that the control device 10 indirectly controls the postures of the elbows, the postures of the shoulders, the position of the head, the posture of the head, and the posture of the spine of the avatar, based additionally on the imaging information on the torso of the user.

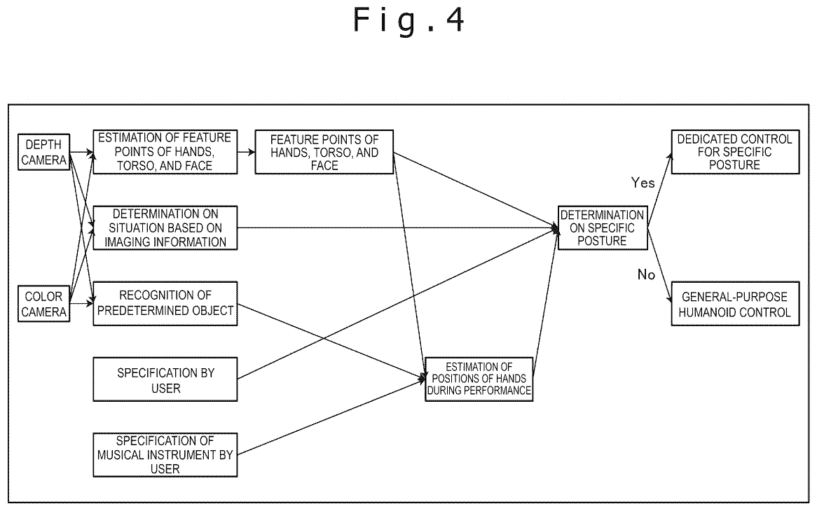

Next, an example of the outline of the processing by the control device 10 for a specific posture (dedicated to a specific posture) will be described with reference to FIG. 4. Examples of the specific posture include a posture in which arms are folded, a posture in which hands are joined together behind the head, and a posture of playing a musical instrument such as a guitar, a piano, or drums.

Based on the imaging information on the user obtained by imaging by the depth camera, the control device 10 estimates feature points such as the hand, torso, and face of the user, estimates a situation represented by the imaging information on the user, and recognizes a predetermined object included in the imaging information on the user. Based on the imaging information on the user obtained by imaging by the color camera, the control device 10 estimates feature points such as the hands, torso, and face of the user, estimates the situation represented by the imaging information on the user, and recognizes a predetermined object included in the imaging information on the user.

When receiving an input of input information in which a musical instrument is specified by the user, the control device 10 estimates positions of o the hands of the user during the performance, based on the estimated feature points such as the hands, torso, and face of the user and on the input information in which the musical instrument is specified by the user. For example, when the user specifies the guitar, the piano, or the drums, the control device 10 calculates position and orientation information on the hands related to positions and orientations of the hands, which corresponds to a position at which the specified musical instrument, among the guitar, the piano, and the drums, is played and also corresponds to positions of the hands of the user. As an example, when the guitar is specified by the user, the control device calculates the position and orientation information on the hands of the user, based on a position of a feature point of the user associated with the guitar, such as the waist of the user.

The control device 10 determines whether the user is in the specific posture, based on at least one of the estimated feature points such as the hands, torso, and face of the user, the estimated situation represented by the imaging information on the user, the input information specified by the user, and the estimated positions of the hands of the user during the performance. For example, when receiving an input of input information in which the guitar is specified by the user, the control device 10 determines whether positions and orientations of the wrists of the user correspond to positions and orientations of the wrists when the user is estimated to be playing the guitar. In this way, the control device 10 determines whether the user is playing the guitar.

When determining that the user is in the specific posture (determined Yes for the specific posture), the control device 10 performs dedicated control for the specific posture on the avatar. For example, the control device 10 imposes a restriction on positions, orientations, or the like of the fingers of the avatar, so that the posture of the avatar corresponds to positions and orientations of the fingers corresponding to the posture in which the arms are folded, or corresponds to positions and orientations of the fingers corresponding to the posture of playing the guitar.

When determining that the user is not in the specific posture (determined No for the specific posture), the control device 10 performs general-purpose control, which is not the control for the specific posture, on the avatar. For example, the control device 10 performs general-purpose humanoid control for controlling the posture of the avatar so as to be a posture corresponding to the posture of the user.

Next, an example of the configuration of the control device 10 will be described with reference to FIG. 1. In the example depicted in FIG. 1, the control device 10 includes an input reception section 11, a camera 12, a control unit 13, a display section 14, and a storage section 15.

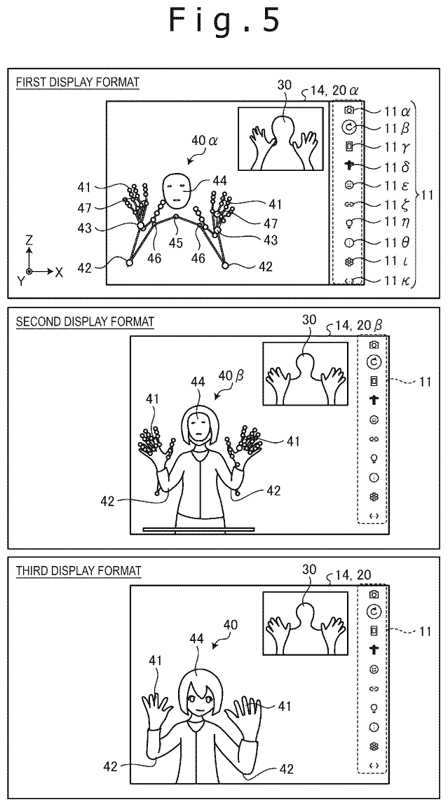

The input reception section 11 receives input information from the user. Examples of the input reception section 11 include a user interface (UI) such as a button displayed on a touch panel. An example of reception of the input information by the input reception section 11 will be described below with reference to FIG. 5. FIG. 5 is a diagram for explaining an example of the control device.

In the example depicted in a diagram of a first mode in FIG. 5, the input reception section 11 is a toolbar including a camera mode switching button 11α, a posture reset button 11β, an application setting button 11γ, a model control setting button 11δ, a facial expression setting button 11ε, a virtual reality model (VRM) setting button 11ζ, a light setting button 11η, an application information button 11Θ, an operation function setting button 11ι, and a UI mode switching button 11κ.

The camera mode switching button 11α is a menu button for switching a mode of the camera 12 described later, in accordance with the input information received from the user. For example, the camera mode switching button 11α changes an orientation and resolution of the camera 12, in accordance with the input information from the user.

The posture reset button 11β is a menu button for resetting the posture of the avatar of the user to a predetermined posture, in accordance with the input information received from the user.

The application setting button 11γ is a menu button for setting a screen, display, or the like of an application executed by the control device 10, such as an application suitable for AR or a VTuber, in accordance with the input information received from the user. For example, the application setting button 11γ is a menu button for causing a display control unit (control unit) 135 described later to switch the display format of a screen 20α depicted in the diagram of a first display format, a screen 203 depicted in the diagram of a second display format, and a screen 20 depicted in the diagram of a third display format in FIG. 5, in accordance with the input information.

As an example, the application setting button 11γ receives, from the user 30, an input of input information for displaying the first display format. The first display format is a format in which an avatar 40α is displayed that reflects a mesh corresponding to skeletal structures of hands of the user 30, which are a result of hand recognition processing performed by a hand recognition processing unit 13111 described later, a skeletal structure of an upper body of the user 30, which is a result of upper body recognition processing performed by an upper body recognition processing unit 13112 described later, and a face of the user 30, which is a result of face recognition processing performed by a face recognition processing unit 13113 described later.

As another example, the application setting button 11γ receives, from the user 30, an input of input information for displaying the second display format. The second display format is a format in which an avatar 40β is displayed that reflects a mesh corresponding to the skeletal structures of the hands of the user 30 and the face of the user 30.

As another example, the application setting button 11γ receives, from the user 30, an input of input information for displaying the third display format. The third display format is a format in which the avatar 40 is displayed that is not reflecting any mesh corresponding to the skeletal structures of the hands of the user 30, the skeletal structure of the upper body of the user 30, and the face of the user 30.

The model control setting button 11δ is a menu button for adjusting values set for a VRM model, which is an example of the avatar of the user, for example, a position of the VRM model and a position of a waist, in accordance with the input information received from the user.

The facial expression setting button 11ε is a menu button for performing settings for controlling a facial expression and parts of the face of the VRM model, in accordance with the input information received from the user.

The VRM setting button 113 is a menu button for changing the VRM model displayed on the screen, in accordance with the input information received from the user.

The light setting button 11ζ is a menu button for setting light to be applied to the VRM model, in accordance with the input information received from the user.

The application information button 11Θ is a menu button for displaying information on an application executed by the control device 10, in accordance with the input information received from the user.

The operation function setting button 11ι is a menu button for performing settings for a function related to an operation of the application executed by the control device 10, in accordance with the input information received from the user. For example, the operation function setting button 11ι changes the execution speed of the application, in accordance with the input information received from the user.

The UI mode switching button 11κ is a menu button for switching a menu of the toolbar, which is the input reception section 11, in accordance with the input information received from the user. An example of the switching of the menu by the UI mode switching button 11κ will be described below with reference to FIG. 5. FIG. 5 is a diagram for explaining an example of the control device. For example, when being touched by the user, the UI mode switching button 11κ switches the menu bar from a menu bar in a developer mode suitable for development of an application depicted in the diagram of the first display format in FIG. 5, to a menu bar in a distributor mode, described later, suitable for distribution by a VTuber or the like.

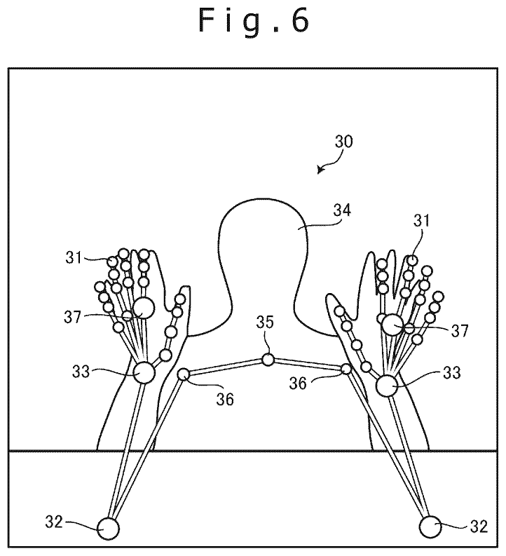

The camera 12 acquires imaging information obtained by imaging the user. In the example depicted in FIG. 1, the camera 12 includes a depth camera 121 and a color camera 122. The depth camera 121 images the hands, the torso, and the face of the user, and acquires imaging information on hands, a torso, and a face, obtained by imaging the torso, and the face of the user. An example of the imaging by the depth camera 121 will be described below with reference to FIG. 6. FIG. 6 is a diagram for describing an example of the control device. As depicted in FIG. 6, the depth camera 121 acquires imaging information on an upper body obtained by imaging the upper body of the user 30, the upper body including the hands (fingers 31, wrists 33, and centers of hands 37), the torso (a neck 35 and shoulders 36), and the face of the user 30. The color camera 122 images the face of the user and acquires imaging information on a face obtained by imaging the face of the user. The color camera 122 may acquire imaging information on hands, a torso, and a face obtained by imaging the hands and the torso of the user.

The control unit 13 controls the whole of the control device 10. The control unit 13 is constituted by, for example, one or more processors including an internal memory that stores control data and a program defining each of processing procedures, and the processor executes each processing by using the program and the internal memory. Examples of the control unit 13 include electronic circuits such as a central processing unit (CPU), a micro processing unit (MPU), and a graphics processing unit (GPU), and integrated circuits such as an application specific integrated circuit (ASIC) and a field programmable gate array (FPGA). In the example depicted in FIG. 1, the control unit 13 includes an acquisition unit 131, a calculation unit 132, a determination unit 133, a model control unit (control unit) 134, and a display control unit 135.

The acquisition unit 131 acquires position information on the fingers and the elbows related to the positions of the fingers and the elbows of the user. The acquisition unit 131 may further acquire position information on at least one of the hands, the shoulders, the elbows, the head, and the neck related to the position of at least one of the hands, the shoulders, the elbows, the head, the face, and the neck of the user. As an example, the acquisition unit 131 acquires position information on the hands and the elbows. As another example, the acquisition unit 131 acquires position information on the hands, the shoulders, the neck, and the elbows related to the positions of the hands, the shoulders, the neck, and the elbows. The acquisition unit 131 may acquire position information on the hands including position information on the wrists related to the positions of the wrists of the user.

A method of acquiring the position information on the fingers and the elbows by the acquisition unit 131 is not particularly limited, but the acquisition unit 131 may acquire the position information on the fingers and the elbows, based on depth information on the fingers and the elbows, for example. The acquisition unit 131 may acquire the position information on at least one of the hands, the shoulders, the elbows, the head, and the neck of the user, based on feature points of the upper body in depth information on the upper body of the user. The acquisition unit 131 may further acquire imaging information on the user obtained by imaging the user. For example, the acquisition unit 131 acquires imaging information on the upper body of the user obtained by imaging the upper body of the user with the depth camera 121. The acquisition unit 131 acquires position information on the fingers, the elbows, the hands, the shoulders, the elbows, the head, and the neck, based on the feature points of the upper body in the depth information on the upper body of the user, which is included in the acquired imaging information. Before describing the configuration of the acquisition unit 131, an overview of the acquisition unit 131 will be described below with reference to FIG. 6.

The acquisition unit 131 acquires imaging information on the hands, the torso, and the face obtained by imaging, with the depth camera 121, the upper body (bust top) of the user 30 positioned above the waist, such as the hands, the torso, the face, and the like of the user 30. The acquisition unit 131 acquires position information on the fingers 31, the elbows 32, the wrists 33, the head 34, the face, the neck 35, and the shoulders 36 of the user 30, based on depth information included in the imaging information on the hands, the torso, and the face. As an example, the acquisition unit 131 allocates the fingers 31, the elbows 32, the wrists 33, the head 34, the neck 35, and the shoulders 36 of the user 30, to each depth in a predetermined range indicated by the depth information included in the imaging information on the hands, the torso, and the face, and acquires position information thereof. The acquisition unit 131 may further acquire posture information on the head 34 of the user 30 using a similar method.

The acquisition unit 131 may acquire the position information on the fingers and the elbows, based on the imaging information on the fingers and the elbows obtained by imaging the fingers and the elbows by the color camera 122, or may acquire the position information on the fingers and the elbows based on the input information on the user received by the input reception section 11. In this case, the acquisition unit 131 acquires the position information on the fingers and the elbows based on color information included in imaging information on the fingers and the elbows obtained by imaging the fingers and the elbows by the color camera 122. The acquisition unit 131 acquires, as the position information on the fingers and the elbows, positions of the fingers and the elbows of the user indicated by the input information on the user received by the input reception section 11.

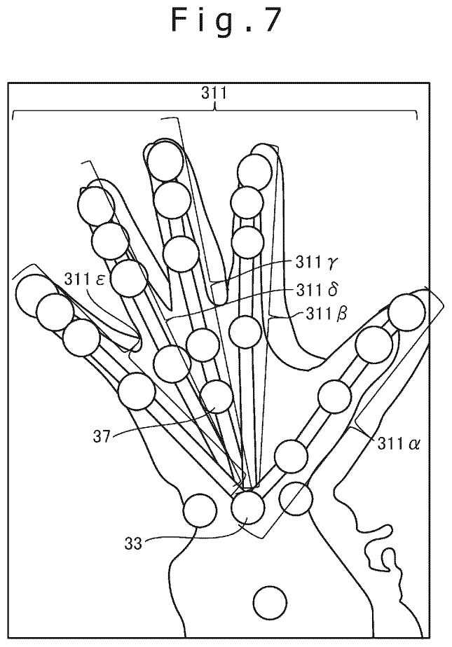

The acquisition unit 131 may further acquire length information on the fingers related to lengths of the fingers. As a result, the acquisition unit 131 can acquire the position information on the fingers with higher accuracy. The acquisition unit 131 may further acquire the length information on the fingers based on the imaging information on the fingers obtained by imaging the fingers by the depth camera 121, and the acquisition unit 131 may further acquire the length information on the fingers indicated by the input information on the user received by the input reception section 11. An example of the acquisition of the length information on the fingers by the acquisition unit 131 will be described below with reference to FIG. 7. FIG. 7 is a diagram for explaining an example of the control device.

In the example depicted in FIG. 7, the acquisition unit 131 acquires thumb length information 311α, index finger length information 311β, middle finger length information 311γ, ring finger length information 311δ, and little finger length information 311ε based on the imaging information on the fingers obtained by imaging the fingers by the depth camera 121. Specifically, the acquisition unit 131 acquires, as the thumb length information 311α, information related to a length from the wrist 33 of the user to the tip of the thumb in the imaging information on the fingers. The acquisition unit 131 acquires information related to the length from the wrist 33 to the tip of the index finger, as the index finger length information 311β. The acquisition unit 131 acquires information related to the length from the wrist 33 to the tip of the middle finger, as the middle finger length information 311γ. The acquisition unit 131 acquires information related to the length from the wrist 33 to the tip of the ring finger, as the ring finger length information 311δ. The acquisition unit 131 acquires information related to the length from the wrist to the tip of the little finger, as the little finger length information 311ε.

In the example depicted in FIG. 7, the acquisition unit 131 acquires the length information on each of the fingers of the right hand of the user, but may also acquire the length information on each of the fingers of the left hand, may acquire the length information on some of the fingers, or may further acquire the length information on joints of the fingers related to the length between the joints of the fingers. By further acquiring the length information on the joints of the fingers related to the length between the joints of the fingers, and by further acquiring the length information on the joints of the fingers related to the length between the joints of the fingers, the acquisition unit 131 can acquire the position information on the fingers with higher accuracy.

The acquisition unit 131 may further acquire posture information related to the specific posture. The specific posture refers to a posture that the user intentionally takes.

Examples of the specific posture include a posture in which the user is playing a musical instrument, a posture in which the user is holding a basin in a hand, a posture in which the user is clapping hands, a posture in which only thumbs of both hands are closed, and a posture of the upper body of the user, in which it is difficult to estimate the position information on the fingers and the elbows from the imaging information. Examples of the posture in which it is difficult to estimate the position information on the fingers and the elbows from the imaging information include a posture in which the arms are folded and a posture in which the hands are joined together behind the head. Therefore, the acquisition unit 131 may acquire, as the posture information related to the specific posture, posture information related to the posture in which the user is playing the musical instrument, the posture in which the arms are folded, or the posture in which the hands are joined together behind the head. For example, the acquisition unit 131 acquires posture information related to a posture in which the user is playing the guitar, the piano, or the drums.

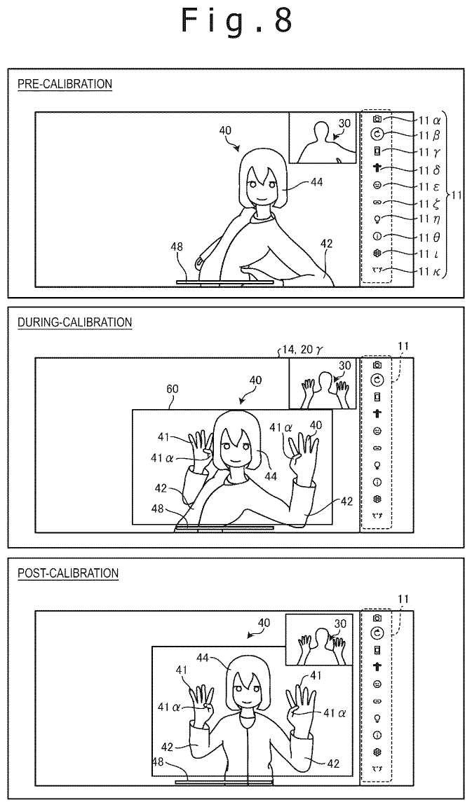

The acquisition unit 131 may further acquire calibration information for calibrating the posture of the avatar. An example of the acquisition of the calibration information by the acquisition unit 131 will be described below with reference to FIG. 8. FIG. 8 is a diagram for explaining an example of the control device. A pre-calibration diagram in FIG. 8 is a diagram depicting a state in which the imaging information on the face of the user is lost as a result of the camera 12 having been moved by the user, and the position information on the face has not been acquired by the acquisition unit 131. Here, in the example depicted in FIG. 8, the camera 12 images only the upper body of the user, and does not image the waist of the user from the beginning. Therefore, the position of the waist cannot be recognized from the imaging information of the camera 12. Therefore, as depicted in the pre-calibration diagram in FIG. 8, even though the user is sitting upright, the waist of the avatar 40 may be positioned inappropriately and the body of the avatar 40 may be twisted.

Thus, the acquisition unit 131 acquires calibration information for calibrating the position of the waist of the avatar 40, so that the waist is positioned vertically below the head of the avatar 40. In this case, the acquisition unit 131 acquires at least one of input information related to the calibration information and posture information related to the specific posture of the user or the avatar 40.

For example, the acquisition unit 131 acquires the calibration information when an input of input information is received by the posture reset button 11β, which is a button for updating the posture depicted in the pre-calibration diagram in FIG. 8. In the example depicted in a during-calibration diagram in FIG. 8, the acquisition unit 131 acquires, instead of or in addition to the input information, posture information indicating that the user or the avatar 40 is in a posture in which only thumbs 41α are closed in both hands, as the calibration information. In this case, the acquisition unit 131 may acquire the calibration information, when the posture information indicating that the user or the avatar 40 is in the posture in which only the thumbs 41α are closed in both hands is acquired over a predetermined time period or longer. The predetermined time period over which the acquisition unit 131 acquires the posture information indicating that the user or the avatar 40 is in the specific posture, such as the posture in which only the thumbs 41α are closed in both hands, is not particularly limited, and may be, for example, one second or longer, or two seconds or longer.

In this way, the acquisition unit 131 may acquire the calibration information, when the posture information indicating that the user or the avatar 40 is in the specific posture is acquired. The acquisition unit 131 may acquire the calibration information, when the posture information indicating that the user or the avatar 40 is in the posture in which only the thumbs 41α are closed in both hands is acquired. The acquisition unit 131 may acquire the calibration information, when the posture information is acquired over the predetermined time period or longer. The acquisition unit 131 may acquire the calibration information, when the posture information is acquired over two seconds or longer.

If the time period over which the posture information is acquired by the acquisition unit 131 is a short time period, there is a possibility that the posture of the avatar has become that posture simply by accident. In contrast, when the time period over which the posture information is acquired by the acquisition unit 131 is the predetermined time period, the posture of the avatar is more likely to be a specific posture for calibration. In particular, when the time period over which the posture information is acquired by the acquisition unit 131 is two seconds or longer, the posture of the avatar is even more likely to be the specific posture for calibration. Therefore, when acquiring the posture information over the predetermined time period or longer, the acquisition unit 131 can acquire the calibration information more accurately than in a case of acquiring the posture information only over a short time period. Further, when acquiring the posture information over two seconds or longer, the acquisition unit 131 can acquire the calibration information more accurately.

Next, the configuration of the acquisition unit 131 will be explained with reference to FIG. 1. In the example depicted in FIG. 1, the acquisition unit 131 includes a recognition processing unit 1311. The recognition processing unit 1311 performs processing of recognizing the hands, the upper body, and the face of the user, based on the imaging information acquired by the camera 12, and acquires position information on the hands, the upper body, and the face related to positions of the recognized hands, upper body, and face of the user. In the example depicted in FIG. 1, the recognition processing unit 1311 includes a hand recognition processing unit 131111, an upper body recognition processing unit 13112, and a face recognition processing unit 13113.

The hand recognition processing unit 131111 performs processing of recognizing the hands of the user, based on imaging information on the hands obtained by imaging the hands of the user by the depth camera 121. For example, the hand recognition processing unit 131111 performs processing of recognizing a skeletal structure of each joint of the hands of the user, such as the wrists 33 and the centers of hands 37, as depicted in FIG. 7, and acquires position information on the recognized hands.

The upper body recognition processing unit 13112 performs processing of recognizing the upper body of the user, based on imaging information on the torso obtained by imaging the torso of the user by the depth camera 121. For example, as depicted in FIG. 6, the upper body recognition processing unit 13112 performs the processing of recognizing the upper body of the user 30, based on imaging information on the torso obtained by imaging the torso of the user by the depth camera 121. In this case, the upper body recognition processing unit 13112 performs processing of recognizing skeletal structures of the fingers 31, the elbows 32, the wrists 33, the head 34, the neck 35, and the shoulders 36 of the user 30, based on feature points in depth information included in the imaging information on the torso obtained by imaging the torso of the user by the depth camera 121. The upper body recognition processing unit 13112 may perform processing of recognizing the centers of hands 37 of the user 30. The upper body recognition processing unit 13112 acquires position information on the fingers 31, the elbows 32, the wrists 33, the head 34, the neck 35, and the shoulders 36 of the user 30 recognized.

The face recognition processing unit 13113 performs processing of recognizing the face of the user, based on imaging information on the face obtained by imaging the face of the user by the depth camera 121 and on imaging information on the face obtained by imaging the face of the user by the color camera 122. For example, as depicted in FIG. 6, the face recognition processing unit 13113 performs processing of extracting the face of the user 30 from the head 34 of the user 30, based on depth information included in the imaging information on the face obtained by imaging the face of the user 30 by the depth camera 121, and of recognizing a skeletal structure of the face of the user 30. The face recognition processing unit 13113 acquires position information on the recognized face of the user 30.

The calculation unit 132 calculates the elbow joint orientation information related to orientations of joints of the elbows, based on the position information on the fingers and the elbows acquired by the acquisition unit 131. The calculation unit 132 may further calculate the finger joint orientation information related to orientations of joints of the fingers, based on the position information on the fingers. The calculation unit 132 may calculate the elbow joint orientation information, based on the position information on at least one of the hands, the shoulders, the elbows, the head, and the neck acquired by the acquisition unit 131. The calculation unit 132 may calculate the joint orientation information on at least one of the hands, the shoulders, the elbows, and the neck and/or the posture information on the head, based on the position information on at least one of the hands, the shoulders, the elbows, the head, and the neck of the user. For example, the calculation unit 132 calculates the elbow joint orientation information, based on the position information on the hands and the elbows acquired by the acquisition unit 131. In this case, the calculation unit 132 calculates the elbow joint orientation information by performing IK processing on the position information on the hands and the elbows acquired by the acquisition unit 131. Specifically, the calculation unit 132 calculates, as the elbow joint orientation information, the rotation angles of the joints of the elbows at which positions of the hands and the elbows indicated by the position information on the hands and the elbows acquired by the acquisition unit 131 become natural.

An example of the calculation by the calculation unit 132 will be explained below with reference to FIG. 6. In the example depicted in FIG. 6, the calculation unit 132 calculates joint orientation information on the hands (the fingers 31, the wrists 33, and the centers of hands 37), the elbows 32, the shoulders 36, and the shoulders 36 and the posture information on the head 34, based on the position information on the hands (the fingers 31, the wrists 33, and the centers of hands 37), the elbows 32, the head 34, the shoulders 36, and the shoulders 36 included in the imaging information on the upper body of the user 30 obtained by imaging by the depth camera 121 depicted in FIG. 6. In this case, the calculation unit 132 calculates the joint orientation information (rotation angles) of the hands (the fingers 31, the wrists 33, and the center of hands 37), the elbows 32, the shoulders 36, and the shoulders 36 and the posture information (rotation angle) of the head 34, based on the position information on the upper body of the user 30 acquired by the upper body recognition processing unit 13112 in the acquisition unit 131. The calculation unit 132 may calculate the joint orientation information (rotation angles) of the hands, based on the position information on the hands (the fingers 31, the wrists 33, and the centers of hands 37) acquired by the hand recognition processing unit 13111 instead of the upper body recognition processing unit 13112.

The calculation unit 132 may calculate the joint orientation information on the fingers and the elbows, based additionally on the length information on the fingers acquired by the acquisition unit 131. In this way, the joint orientation information on the fingers and the elbows can be calculated with higher accuracy. In the example depicted in FIG. 7, the calculation unit 132 calculates the orientation information on the fingers and the elbows, based additionally on the thumb length information 311α, the index finger length information 311β, the middle finger length information 311γ, the ring finger length information 311δ, and the little finger length information 311ε, which are acquired by the depth camera 121. As an example, the calculation unit 132 compares the length information on each of the fingers, length information for each orientation of each of the fingers, and elbow joint orientation information associated with the length information for each orientation of each of the fingers, and calculates joint orientation information on each of the fingers and the elbow joint orientation information.

The calculation unit 132 may further calculate the position information on at least one of the hands, the shoulders, the elbows, the head, and the neck of the avatar, based on the position information on at least one of the hands, the shoulders, the elbows, the head, and the neck of the user.

For example, the calculation unit 132 calculates position information on the hands (fingers 41, wrists 43, and centers of hands 47), elbows 42, wrists 43, a head 44, a neck 45, and shoulders 46 of the avatar 40α depicted in the diagram of the first display format in FIG. 4, based on the position information on the hands (the fingers 31, the wrists 33, and the centers of hands 37), the elbows 32, the wrists 33, the head 34, the neck 35, and the shoulders 36 of the user 30 depicted in FIG. 6. The calculation unit 132 may calculate the posture information on the head of the avatar, based on the posture information on the head instead of the position information on the head. The calculation unit 132 may further calculate joint orientation information on at least one of the hands, the shoulders, the elbows, and the neck and/or the posture information on the head of the avatar, based on the position information on at least one of the hands, the shoulders, the elbows, the head, and the neck of the user or the avatar.

The calculation unit 132 may estimate the shoulder joint orientation information related to the orientations of the joints of the shoulders of the avatar with respect to a direction directly facing the screen displayed on the control device 10 and the vertical direction, based on the position information on the hands, the shoulders, the neck, and the elbows acquired by the acquisition unit 131. For example, the calculation unit 132 estimates joint orientation information on the shoulders 46 of the avatar 40 with respect to the Y-axis direction and the Z-axis direction depicted in FIG. 5, based on the position information on the hands (the fingers 31, the wrists 33, and the centers of hands 37), the shoulders 36, the neck 35, and the elbows 32, which are depicted in FIG. 6, acquired by the acquisition unit 131.

When the position information on the face has not been acquired by the acquisition unit 131, the calculation unit 132 may estimate the position information on the face, based on the position information on the hands, the shoulders, the neck, and the elbows acquired by the acquisition unit 131. For example, when the imaging information on the face of the user is lost, the calculation unit 132 may estimate the position information on the face of the avatar related to the position of the face of the avatar, based on the position information on the hands, the shoulders, the neck, and the elbows. When the position information on the face has not been acquired by the acquisition unit 131, the calculation unit 132 may further estimate the posture information on the face of the avatar related to the posture of the face of the avatar, based on the position information on the hands, the shoulders, the neck, and the elbows.

The determination unit 133 determines whether the user is in the specific posture. The determination unit 133 may determine whether the user is in the specific posture, based on the posture information, the position information on the fingers and the elbows, and the finger and elbow joint orientation information, which are acquired by the acquisition unit 131. The determination unit 133 may determine whether the user is in the specific posture, based additionally on at least one of the situation represented by the imaging information on the user, the predetermined object included in the imaging information on the user, and the input information on the user. Examples of the predetermined object include a musical instrument, a basin, and a marker. As an example, the determination unit 133 determines whether the user is in a posture in which the user is playing a musical instrument, based on a musical instrument included in the imaging information on the user. As another example, the determination unit 133 determines whether the user is in a posture in which the user is playing the guitar, based on input information on the user which specifies posture information related to the posture in which the user is playing the guitar.

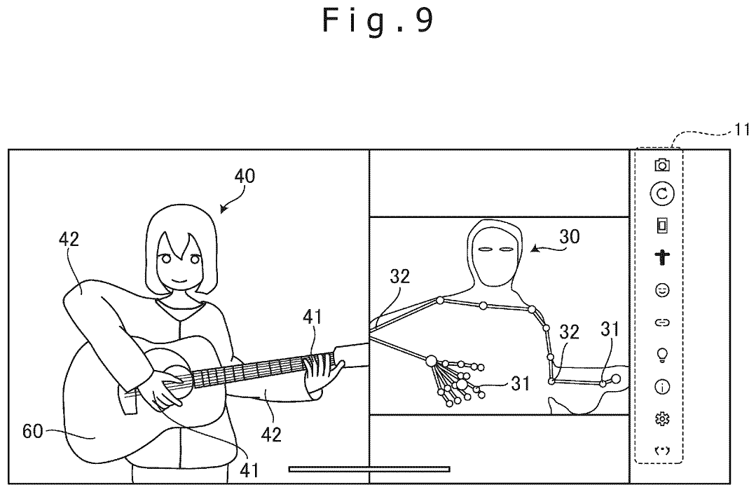

An example of the determination by the determination unit 133 will be explained below with reference to FIG. 9. FIG. 9 is a diagram for explaining an example of the control device. In the example depicted in FIG. 9, the determination unit 133 determines whether the user 30 is in the posture of playing the guitar, based on input information on the user in which the specific posture is specified as the posture information related to the posture in which the user is playing the guitar, the position information on the fingers 31 and the elbows 32, and the joint orientation information on the fingers 31 and the elbows 32, which have been acquired by the acquisition unit 131.

The model control unit 134 controls the posture of the avatar of the user, based on the elbow joint orientation information calculated by the calculation unit 132. The model control unit 134 may control the posture of the avatar, based additionally on the finger joint orientation information calculated by the calculation unit 132. For example, the model control unit 134 controls the postures of the fingers 41 and the elbows 42 of the avatar 40 such as a VRM model depicted in the diagram of the third display format in FIG. 5, based on the joint orientation information on the fingers 31 and the elbows 32 of the user 30, which are depicted in FIG. 6, calculated by the calculation unit 132. The model control unit 134 controls the postures of the fingers and the elbows of the avatar 40 so as to fully track movements of the fingers and the elbows of the user 30 depicted in the diagram of the third display format in FIG. 5.

The model control unit 134 may control the posture of the avatar, based on the elbow joint orientation information on the avatar calculated by the calculation unit 132, instead of the elbow joint orientation information calculated by the calculation unit 132. The model control unit 134 may control the posture of the avatar, based on the joint orientation information on at least one of the hands, the shoulders, the elbows, and the neck of the user or the avatar, and/or the posture information on the head, which are calculated by the calculation unit 132.

An example of the control of the posture of the avatar by the model control unit 134 will be described with reference to FIGS. 5 and 6. In the example depicted in FIG. 6, the upper body recognition processing unit 13112 acquires the position information on the upper body of the user 30 from the imaging information on the torso of the user 30, and the calculation unit 132 calculates the joint orientation information (rotation angles) of the hands (the fingers 31, the wrists 33, and the centers of hands 37), the elbows 32, the neck 35, and the shoulders 36 of the user 30 and the posture information (rotation angle) of the head 34. In this case, the model control unit 134 controls the postures of the hands (the fingers 41, the wrists 43, and the centers of hands 47), the elbows 42, the neck 45, the shoulders 46, and the head 44 of the avatar 40α depicted in the diagram of the first display format in FIG. 5, based on the calculated joint orientation information on the hands, the elbows 32, the neck 35, and the shoulders 36 and the calculated posture information on the head.

The model control unit 134 may control the posture of the avatar that is morphed into 2D (2-Dimensions). For example, as depicted in the diagram of the third display format in FIG. 5, the model control unit 134 controls the posture of the avatar 40 morphed into 2D. The model control unit 134 may control the posture of the avatar morphed into 3D (3-Dimensions) instead of the avatar 40 morphed into 2D, or may control the posture of an avatar for AR (augmented reality) instead of the avatar 40 for VR.

When the calibration information is acquired by the acquisition unit 131, the model control unit 134 may calibrate the posture of the avatar so that the waist is positioned vertically below the head of the avatar corresponding to the position information on the face estimated by the calculation unit 132. For example, as depicted in a post-calibration diagram in FIG. 8, when the calibration information is acquired by the acquisition unit 131, the acquisition unit 131 calibrates the posture of the avatar 40 so that a waist 48 is positioned vertically below the face of the avatar 40 corresponding to the position information on the face estimated by the calculation unit 132.

When the determination unit 133 determines that the user is in the specific posture, the model control unit 134 controls the posture of the avatar so that the posture of the avatar becomes a posture corresponding to the specific posture. For example, when the determination unit 133 determines that the user is in the posture in which the user is playing the musical instrument, the posture in which the arms are folded, or the posture in which the hands are joined together behind the head, the model control unit 134 controls the posture of the avatar so that the posture of the avatar becomes a posture corresponding to the posture determined by the determination unit 133 among the posture in which the user is playing the musical instrument, the posture in which the arms are folded, and the posture in which the hands are joined together behind the head. As an example, when the determination unit 133 determines that the user is in a posture in which the user is playing the guitar, the piano, or the drums, the model control unit 134 controls the posture of the avatar so that the posture of the avatar becomes a posture corresponding to the posture in which the user is playing the guitar, the piano, or the drums. An example of the control of the posture of the avatar by the model control unit 134 will be explained below with reference to FIG. 9. FIG. 9 is a diagram for explaining an example of the control device.

When the determination unit 133 determines that a guitar mode is applied in which the user 30 is in a posture of playing the guitar (singing while playing the guitar), the model control unit 134 controls the posture of the avatar 40 to be a posture corresponding to the posture of playing a guitar 60.

In the example depicted in FIG. 9, the determination unit 133 determines that the guitar mode is applied when the finger 31 of the left hand of the user 30 is positioned in a region specified in advance as a region near the neck of the guitar, and the palm of the left hand of the user 30 is in an orientation directly facing the torso of the user 30. When the determination unit 133 determines that the guitar mode is applied, the model control unit 134 switches the mode to the guitar mode and controls the posture of the avatar 40 to be a posture corresponding to the posture of playing the guitar 60. In this case, the model control unit 134 controls the posture of the avatar 40 so that the fingers 41 of both hands of the avatar 40 are positioned on the neck of the guitar 60 and positioned so as to correspond to the positions of the fingers 31 of both hands of the user 30. Further, the model control unit 134 controls the posture of the avatar 40 so that the elbow 42 of the left arm of the avatar 40 is in a position corresponding to the position of the elbow 32 of the left arm of the user 30. In the example depicted in FIG. 9, when the mode is switched to the guitar mode, the model control unit 134 controls the posture of the avatar 40 so that the position of the elbow 42 of the right arm is higher than the position of the elbow 32 of the right arm of the user 30 in the vertical direction. In this way, the model control unit 134 controls the posture of the avatar 40 in the guitar mode, for example, in a state where the position of the elbow 42 of the right arm of the avatar 40 can be adjusted to any position in accordance with the input information on the user.

The display control unit 135 controls display by the display section 14. For example, as depicted in the diagram of the third display format in FIG. 5, the display control unit 135 causes the display section 14 to display the screen 20 on which the avatar 40 of the user 30, which is a VRM model, is drawn.

Further, as depicted in the diagram of the first display format in FIG. 5, when an input of input information for displaying the screen 20α of the first display format is received from the user 30 by the application setting button 11γ, the display control unit 135 displays the screen 20α on the display section 14. As depicted in the diagram of the second display format in FIG. 5, when an input of input information for displaying the screen 20β of the second display format is received from the user 30 by the application setting button 11γ, the display control unit 135 displays the screen 20β on the display section 14. As depicted in the diagram of the third display format in FIG. 5, when an input of input information for displaying the screen 20 of the third display format is received from the user 30 by the application setting button 11γ, the display control unit 135 displays the screen 20 on the display section 14.

The display control unit 135 may display a range corresponding to an imaging range of the user on the screen on which the avatar is displayed while the calibration is being performed by the model control unit 134. For example, as depicted in the during-calibration diagram in FIG. 8, the display control unit 135 causes the display section 14 to display a range 50 corresponding to the imaging range of the user 30 by the camera 12 on the screen 20γ on which the avatar 40 is displayed, while the calibration is being performed. In this case, the display control unit 135 calculates an extent of the imaging range of the user 30 on the screen 20γ on which the avatar 40 is displayed, using the field of view (FoV), which is the viewing angle, of the camera 12 in the real world and the FoV of a virtual camera in the application. The display control unit 135 causes the display section 14 to display, on the screen 20γ, the calculated range as the range 50.

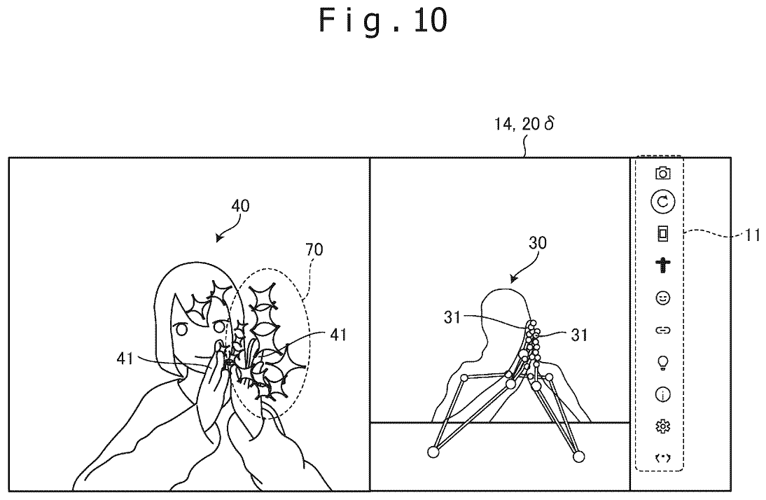

The display control unit 135 may display an effect on the screen, based on the posture of the user or the avatar. For example, when the determination unit 133 determines that the posture of the user is the specific posture, the display control unit 135 displays an effect corresponding to the specific posture on the screen. An example of the display control by the display control unit 135 will be described below with reference to FIG. 10. FIG. 10 is a diagram for explaining an example of the control device. In the example depicted in FIG. 10, the determination unit 133 determines that the posture of the user 30 is a posture of clapping hands, based on the positions of the fingers 31 of the user 30. In this case, the display control unit 135 causes the display section 14 to display a sparkling effect 70 on a screen 20δ in a region near the fingers 41 of both hands of the avatar 40 (within a predetermined range from the fingers 41).

In the example depicted in FIG. 10, the display control unit 135 causes the display section 14 to display the sparkling effect 70 on the screen 20δ, but may cause the display section 14 to display a different effect such as a water effect or a heart effect instead of the sparkling effect 70.

The display section 14 displays various images under the control of the display control unit 135. Examples of the display section 14 include a touch panel of a smartphone.

The storage section 15 stores various types of information such as the position information on the fingers and the elbows and the elbow joint orientation information. Examples of the storage section 15 include a storage device such as a hard disk drive (HDD), a solid state drive (SSD), and an optical disk, and a semiconductor memory capable of rewriting data such as a random access memory (RAM), a flash memory, and a non-volatile static random access memory (NVSRAM). The storage section 15 stores an operating system (OS) and various types of programs executed by the control device 10.

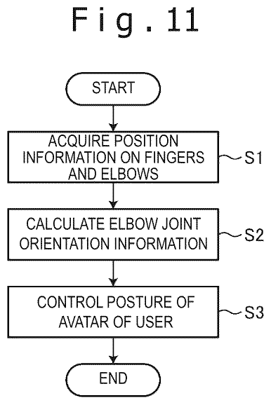

Next, an example of a flow of processing by the control device 10 will be explained with reference to FIG. 11. FIG. 11 is a diagram depicting an example of the flow of processing by the control device.

In step S1, the acquisition unit 131 acquires the position information on the fingers and the elbows related to the positions of the fingers and the elbows of the user.

In step S2, the calculation unit 132 calculates the elbow joint orientation information related to the orientations of the joints of the elbows based on the position information on the fingers and the elbows acquired by the acquisition unit 131.

In step S3, the model control unit 134 controls the posture of the avatar of the user, based on the elbow joint orientation information calculated by the calculation unit 132.

### 2. Example of Hardware Configuration

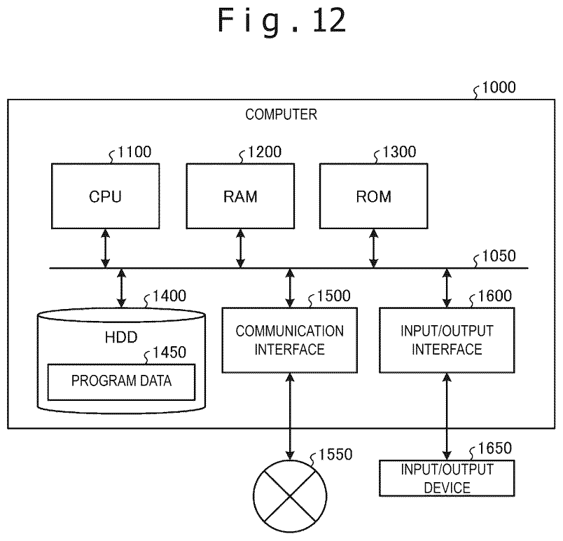

The control device 10 explained above may include a computer. An example will be explained with reference to FIG. 12.

FIG. 12 is a diagram depicting an example of a hardware configuration of a device. A computer 1000 illustrated in the drawing includes a CPU 1100, a RAM 1200, a read only memory (ROM) 1300, an HDD 1400, a communication interface 1500, and an input/output interface 1600. The respective units of the computer 1000 are connected to each other via a bus 1050.

The CPU 1100 operates based on programs stored in the ROM 1300 or the HDD 1400, and controls each of the units. For example, the CPU 1100 deploys the program stored in the ROM 1300 or the HDD 1400 to the RAM 1200, and executes processing corresponding to the various types of programs.

The ROM 1300 stores a boot program such as a basic input output system (BIOS) executed by the CPU 1100 at the time of starting the computer 1000, a program dependent on the hardware of the computer 1000, and the like.

The HDD 1400 is a computer-readable recording medium that non-temporarily records the program executed by the CPU 1100 and data and the like used by the program. Specifically, the HDD 1400 is a recording medium that records a control program, which is an example of program data 1450, for executing each operation according to the present disclosure.

The communication interface 1500 is an interface for the computer 1000 to connect to an external network 1550 (for example, the Internet). For example, the CPU 1100 receives data from another device via the communication interface 1500, and transmits data generated by the CPU 1100 to another device via the communication interface 1500.

The input/output interface 1600 is an interface for connecting an input/output device 1650 and the computer 1000. For example, the CPU 1100 receives data from an input device such as a keyboard or a mouse via the input/output interface 1600. The CPU 1100 also transmits data to an output device such as a display, a speaker, a printer or the like via the input/output interface 1600. Further, the input/output interface 1600 may function as a media interface that reads a program or the like recorded in a predetermined recording medium (medium). Examples of the medium include an optical recording medium such as a digital versatile disc (DVD) or a phase change rewritable disk (PD), a magneto-optical recording medium such as a magneto-optical disk (MO), a tape medium, a magnetic recording medium, a semiconductor memory, and the like.

At least some of the functions of the control device 10 explained above may be realized by, for example, the CPU 1100 of the computer 1000 executing a program loaded onto the RAM 1200. The HDD 1400 stores a program and the like according to the present disclosure. Note that the CPU 1100 reads the program data 1450 from the HDD 1400 to execute the program, but as another example, the CPU 1100 may acquire the program from another device via the external network 1550.

### 3. Example of Effect

The technique described above is specified as follows, for example. One of the disclosed techniques is the control device 10. As described above with reference to FIGS. 1 to 11 and the like, the control device 10 includes the acquisition unit 131 that acquires the position information on the fingers and the elbows related to the positions of the fingers and the elbows of the user, the calculation unit 132 that calculates the elbow joint orientation information related to the orientations of the joints of the elbows, based on the position information on the fingers and the elbows acquired by the acquisition unit 131, and the model control unit 134 that controls the posture of the avatar of the user, based on the elbow joint orientation information calculated by the calculation unit 132. In this way, the control device 10 controls the posture of the avatar of the user, based on the elbow joint orientation information. For example, the control device 10 controls the posture of the avatar so that the fingers, which are the tip portions of the arms of the avatar of the user, are positioned at natural positions in accordance with the orientations of the joints of the elbows. In this way, the control device 10 can control the posture of the avatar with high accuracy.

As described above with reference to FIGS. 1 to 11 and the like, the calculation unit 132 may further calculate the finger joint orientation information related to the orientations of the joints of the fingers, based on the position information on the fingers, and the model control unit 134 may control the posture of the avatar, based additionally on the finger joint orientation information calculated by the calculation unit 132. In this way, the posture of the avatar of the user can be controlled with higher accuracy.

As described above with reference to FIGS. 1 to 11 and the like, the acquisition unit 131 may acquire the position information on the fingers and the elbows, based on the depth information on the fingers and the elbows. In this way, the control device 10 can acquire the position information on the fingers and the elbows with higher accuracy than in a case of acquiring the position information on the fingers and the elbows, based on the color information included in the imaging field information on the fingers and the elbows in which the fingers and the elbows are imaged by the color camera.

As described above with reference to FIGS. 1 to 11 and the like, the acquisition unit 131 may further acquire the position information on at least one of the hands, the shoulders, the elbows, the head, and the neck related to the position of at least one of the hands, the shoulders, the elbows, the head, the face, and the neck of the user, and the calculation unit 132 may calculate the elbow joint orientation information, based additionally on the position information on at least one of the hands, the shoulders, the elbows, the head, and the neck acquired by the acquisition unit 131. In this way, the control device 10 can calculate the elbow joint orientation information with higher accuracy.

As described above with reference to FIGS. 1 to 11 and the like, the acquisition unit 131 may acquire the position information on at least one of the hands, the shoulders, the elbows, the head, and the neck, based on the feature point of the upper body in the depth information on the upper body of the user. In this way, the control device 10 can efficiently acquire the position information on at least one of the hands, the shoulders, the elbows, the head, and the neck.

As described above with reference to FIGS. 1 to 11 and the like, the acquisition unit 131 may acquire the position information on the hands and the elbows, and the calculation unit 132 may calculate the elbow joint orientation information, based on the position information on the hands and the elbows acquired by the acquisition unit 131. In this way, the control device 10 can calculate the elbow joint orientation information with higher accuracy.

As described above with reference to FIGS. 1 to 11 and the like, the calculation unit 132 may calculate the elbow joint orientation information by performing IK processing on the position information on the hands and the elbows acquired by the acquisition unit 131. In this way, the control device 10 calculates the rotation angles of the joints of the elbows by the IK processing, and thus can calculate the elbow joint orientation information with higher accuracy.

As described above with reference to FIGS. 1 to 11 and the like, the acquisition unit 131 may further acquire the position information on the hands, the shoulders, the neck, and the elbows related to the positions of the hands, the shoulders, the neck, and the elbows, and the calculation unit 132 may estimate the shoulder joint orientation information related to the orientations of the joints of the shoulders of the avatar with respect to the direction directly facing the screen displayed on the control device 10 and to the vertical direction, based on the position information on the hands, the shoulders, the neck, and the elbows acquired by the acquisition unit 131. In this way, the control device 10 can estimate the shoulder joint orientation information on the avatar with respect to a plurality of directions with high accuracy.

As described above with reference to FIGS. 1 to 11 and the like, when the position information on the face related to the position of the face of the user is not acquired by the acquisition unit 131, the calculation unit 132 may estimate the position information on the face, based on the position information on the hands, the shoulders, the neck, and the elbows acquired by the acquisition unit 131. The control device 10 can estimate the position information on the face even when the imaging information on the face including the position information on the face is lost.

As described above with reference to FIGS. 1 to 11 and the like, the acquisition unit 131 may further acquire the calibration information for calibrating the posture of the avatar, and when the calibration information is acquired by the acquisition unit 131, the model control unit 134 may calibrate the posture of the avatar so that the waist is positioned vertically below the head of the avatar corresponding to the position information on the face estimated by the calculation unit 132.

Normally, the user such as a VTuber performs a live commentary or the like in a state where the upper body is imaged, and thus the waist is often not imaged. Therefore, when the position of the camera 12 is shifted by the user, the position of the waist of the avatar may be shifted. In particular, when the control device 10 including the camera 12 is a small device such as a smartphone, the user may inevitably move away from the imaging range of the camera 12. In contrast, the control device 10 performs the calibration when the acquisition unit 131 acquires, as the calibration information, input information related to the calibration information, the posture information related to the specific posture of the posture of the user or the avatar, or the like, thereby adjusting the position of the waist of the avatar.

As described above with reference to FIGS. 1 to 11 and the like, the acquisition unit 131 may acquire the calibration information, when posture information is acquired indicating that the user or the avatar is in the specific posture. In this way, the control device 10 performs the calibration when the acquisition unit 131 acquires, as the calibration information, the posture information indicating that the posture of the user or the avatar is the specific posture such as the posture in which the thumbs are closed, thereby adjusting the position of the waist of the avatar.

As described above with reference to FIGS. 1 to 11 and the like, the acquisition unit 131 may acquire the calibration information, when the posture information is acquired over the predetermined time period or longer. If the time period over which the posture information is acquired by the acquisition unit 131 is a short time period, there is a possibility that the posture of the avatar has become that posture simply by accident. In contrast, when the time period over which the posture information is acquired by the acquisition unit 131 is the predetermined time period, the posture of the avatar is more likely to be a specific posture for calibration. Therefore, when the posture information is acquired by the acquisition unit 131 over the predetermined time period or longer, the control device 10 can acquire the calibration information more accurately than in a case of acquiring the posture information over a short time period.

As described above with reference to FIGS. 1 to 11 and the like, the acquisition unit 131 may acquire the calibration information, when the posture information is acquired for two seconds or longer. When the time period over which the posture information is acquired by the acquisition unit 131 is two seconds or longer, the posture of the avatar is more likely to be the specific posture for calibration, and thus the control device 10 can acquire the calibration information more accurately.

As described above with reference to FIGS. 1 to 11 and the like, during the calibration by the model control unit 134, the display control unit 135 may display the range corresponding to the imaging range of the user on the screen on which the avatar is displayed.

When the user such as a VTuber does not want to show his/her face, it is not preferable that the imaging information on the user is displayed on the screen in a state where the face of the user is clearly recognizable by the camera. For example, if the imaging information on the user from the camera is displayed in the same region as for the avatar, and thus the imaging information on the user is clearly displayed on the screen, the imaging range of the user becomes recognizable, but this may be difficult in some cases. If the imaging range of the user is not recognizable, the imaging information on the hands or the face of the user is more easily lost. Therefore, it is desired that the imaging range of the user be recognizable so that it is possible to recognize that the user is centered in relation to the camera.

On the other hand, the camera 12 in the real world has a horizontal angle of about 60 degrees, whereas the angle of view of the virtual camera in the CG world in which the avatar is imaged is often as narrow as about 30 degrees. Thus, the imaging range of the user is not usually reflected as it is, in an image in which the avatar is displayed. This is because, when the angle of view is set to be the same as that of the camera 12 in the real world, distortion occurs as in a fish-eye camera, and the shape of the avatar looks strange.

In contrast, the control device 10 displays the range corresponding to the imaging range of the user on the screen on which the avatar is displayed. In this way, the control device 10 displays, on the screen, a range corresponding to the imaging range of the user in which the avatar does not look strange, and causes the imaging range of the user to be recognizable. Further, the control device 10 displays the range corresponding to the imaging range of the user on the screen on which the avatar is displayed, only during the calibration. In this way, the control device 10 can cut out the avatar without difficulty when the avatar is cut out using a green screen or the like, compared with a case where the range corresponding to the imaging range of the user is always displayed on the screen on which the avatar is displayed.

As described above with reference to FIGS. 1 to 11 and the like, the control device 10 further includes the determination unit 133 that determines whether the user is in the specific posture, and when the determination unit 133 determines that the user is in the specific posture, the model control unit 134 controls the posture of the avatar to be a posture corresponding to the specific posture. In this way, even when the user is in the specific posture, the control device 10 can reduce the control that, due to erroneous recognition, is not appropriate in terms of the skeletal structure by imposing a restriction on the positions, the orientations, or the like of the fingers of the avatar, so that the posture corresponds to the specific posture.

As described above with reference to FIGS. 1 to 11 and the like, the acquisition unit 131 may further acquire the posture information related to the specific posture, and the determination unit 133 may determine whether the user is in the specific posture, based on the posture information, the position information on the fingers and the elbows, and the joint orientation information on the fingers and the elbows, which are acquired by the acquisition unit 131. In this way, the control device 10 can prevent the posture of the avatar from being unnecessarily controlled, when the positions of the fingers and the elbows and the orientations of the joints of the fingers and the elbows become predetermined positions and orientations, even though the user does not want the posture of the avatar to be controlled into a posture corresponding to the specific posture. As a result, the control device 10 can control the posture of the avatar with higher accuracy when the user is in the specific designation.

As described above with reference to FIGS. 1 to 11 and the like, the acquisition unit 131 may acquire, as the posture information related to the specific posture, the posture information related to the posture in which the user is playing the musical instrument, the posture in which the arms are folded, or the posture in which the hands are joined together behind the head, and when the determination unit 133 determines that the user is in the posture in which the user is playing the musical instrument, the posture in which arms are folded, or the posture in which the hands are joined together behind the head, the model control unit 134 may control the posture of the avatar to be a posture corresponding to the posture determined by the determination unit 133, among the posture in which the user is playing the musical instrument, the posture in which the arms are folded, and the posture in which the hands are joined together behind the head. In this way, the control device 10 can control the posture of the avatar with high accuracy with respect to the posture of playing the musical instrument, the posture in which the arms are folded, and the posture in which the hands are joined together behind the head.

As described above with reference to FIGS. 1 to 11 and the like, the acquisition unit 131 may further acquire the imaging information on the user obtained by imaging the user, and the determination unit 133 may determine whether the user is in the specific posture, based additionally on at least one of the situation represented by the imaging information on the user, the predetermined object included in the imaging information on the user, and the input information on the user. In this way, the control device 10 can determine with higher accuracy whether the user is in the specific posture.

The control method described above with reference to FIGS. 1 to 11 and the like is also one of the disclosed techniques.

The control method is a control method executed by the control device 10, and includes an acquisition step (step S1) of acquiring the position information on the fingers and the elbows related to the positions of the fingers and the elbows of the user, a calculation step (step S2) of calculating the elbow joint orientation information related to the orientations of the joints of the elbows, based on the position information on the fingers and the elbows acquired in the acquisition step, and a control step (step S3) of controlling the posture of the avatar of the user, based on the elbow joint orientation information calculated in the calculation step. With such a control method as well, as described above, the posture of the avatar can be controlled with high accuracy.

The control program described above with reference to FIGS. 1 to 11 and the like is also one of the disclosed techniques. The control program causes the computer 1000 mounted on the control device 10 to execute acquisition processing of acquiring the position information on the fingers and the elbows related to the positions of the fingers and elbows of the user, calculation processing of calculating the elbow joint orientation information related to the orientations of the joints of the elbows, based on the position information on the fingers and the elbows acquired by the acquisition processing, and control processing of controlling the posture of the avatar of the user, based on the elbow joint orientation information calculated by the calculation processing. With such a control program as well, the posture of the avatar can be controlled with high accuracy.

The effects described in the present disclosure are merely examples, and are not limited to the disclosed content. Other effects may be obtained.

Although the embodiments of the present disclosure have been described above, the technical scope of the present disclosure is not limited to the above-described embodiments as they are, and various modifications can be made without departing from the gist of the present disclosure, and different constituent elements may be appropriately combined.

Note that the present technique can also have the following configurations.
(1)

A control device including
an acquisition unit configured to acquire position information on fingers and elbows related to positions of fingers and elbows of a user,
a calculation unit configured to calculate elbow joint orientation information related to orientations of joints of the elbows, based on the position information on the fingers and the elbows acquired by the acquisition unit, and
a control unit configured to control a posture of an avatar of the user, based on the elbow joint orientation information calculated by the calculation unit.
(2)

The control device according to (1), wherein
the calculation unit further calculates finger joint orientation information related to orientations of joints of the fingers, based on the position information on the fingers, and
the control unit controls the posture of the avatar, based additionally on the finger joint orientation information calculated by the calculation unit.
(3)

The control device according to (1) or (2), wherein
the acquisition unit acquires the position information on the fingers and the elbows, based on depth information on the fingers and the elbows.
(4)

The control device according to any one of (1) to (3), wherein
the acquisition unit further acquires position information on at least one of hands, shoulders, elbows, a head, and a neck related to a position of at least one of hands, shoulders, elbows, a head, a face, and a neck of the user, and
the calculation unit calculates the elbow joint orientation information, based additionally on the position information on at least one of the hands, the shoulders, the elbows, the head, and the neck acquired by the acquisition unit.
(5)

The control device according to (4), wherein
the acquisition unit acquires the position information on at least one of the hands, the shoulders, the elbows, the head, and the neck, based on a feature point of an upper body of the user in depth information on the upper body.
(6)

The control device according to (4), wherein
the acquisition unit acquires position information on the hands and the elbows, and
the calculation unit calculates the elbow joint orientation information, based on the position information on the hands and the elbows acquired by the acquisition unit.
(7)

The control device according to (6), wherein
the calculation unit calculates the elbow joint orientation information by performing IK processing on the position information on the hands and the elbows acquired by the acquisition unit.
(8)

The control device according to any one of (1) to (7), wherein
the acquisition unit further acquires position information on hands, shoulders, a neck, and elbows related to positions of the hands, the shoulders, the neck, and the elbows, and
the calculation unit estimates shoulder joint orientation information related to orientations of joints of the shoulders of the avatar with respect to a direction directly facing a screen displayed on the control device and to a vertical direction, based on the position information on the hands, the shoulders, the neck, and the elbows acquired by the acquisition unit.
(9)

The control device according to (8), wherein
when position information on a face related to a position of a face of the user is not acquired by the acquisition unit, the calculation unit estimates the position information on the face, based on the position information on the hands, the shoulders, the neck, and the elbows acquired by the acquisition unit.
(10)

The control device according to (9), wherein
the acquisition unit further acquires calibration information for calibrating the posture of the avatar, and
when the calibration information is acquired by the acquisition unit, the control unit calibrates the posture of the avatar so that a waist of the avatar is positioned vertically below a head of the avatar corresponding to the position information on the face estimated by the calculation unit.
(11)

The control device according to (10), wherein the acquisition unit acquires the calibration information
when posture information is acquired indicating that the user or the avatar is in a specific posture.
(12)

The control device according to (11), wherein the acquisition unit acquires the calibration information
when the posture information is acquired over a predetermined time period or longer.
(13)

The control device according to (12), wherein the acquisition unit acquires the calibration information
when the posture information is acquired over two seconds or more.
(14)

The control device according to any one of (10) to (13), wherein
during calibration by the control unit, the control unit displays a range corresponding to an imaging range of a user on a screen on which the avatar is displayed.
(15)

The control device according to (2) further including
a determination unit configured to determine whether the user is in a specific posture, wherein
when the determination unit determines that the user is in the specific posture, the control unit controls the posture of the avatar to be a posture corresponding to the specific posture.
(16)

The control device according to (15), wherein
the acquisition unit further acquires posture information related to the specific posture, and
the determination unit determines whether the user is in the specific posture, based on the posture information, the position information on the fingers and the elbows, the finger joint orientation information, and the elbow joint orientation information, which are acquired by the acquisition unit.
(17)

The control device according to (16), wherein
the acquisition unit acquires, as the posture information related to the specific posture, posture information related to a posture in which the user is playing a musical instrument, a posture in which arms are folded, or a posture in which hands are joined together behind a head, and
when the determination unit makes a determination that the user is in the posture in which the user is playing the musical instrument, the posture in which the arms are folded, or the posture in which the hands are joined together behind the head, the control unit controls the posture of the avatar to be a posture corresponding to the posture for which the determination has been made by the determination unit, among the posture in which the user is playing the musical instrument, the posture in which the arms are folded, and the posture in which the hands are joined together behind the head.
(18)

The control device according to (17), wherein
the acquisition unit further acquires imaging information on the user obtained by imaging the user, and
the determination unit determines whether the user is in the specific posture, based additionally on at least one of a situation represented by the imaging information on the user, a predetermined object included in the imaging information on the user, and input information on the user.
(19)

A control method executed by a control device, the control method including
an acquisition step of acquiring position information on fingers and elbows related to positions of fingers and elbows of a user,
a calculation step of calculating elbow joint orientation information related to orientations of joints of the elbows based on the position information on the fingers and the elbows acquired in the acquisition step, and
a control step of controlling a posture of an avatar of the user, based on the elbow joint orientation information calculated in the calculation step.
(20)

A control program that causes a computer mounted on a control device to execute
acquisition processing of acquiring position information on fingers and elbows related to positions of fingers and elbows of a user,
calculation processing of calculating elbow joint orientation information related to orientations of joints of the elbows, based on the position information on the fingers and the elbows acquired by the acquisition processing, and
control processing of controlling a posture of an avatar of the user, based on the elbow joint orientation information calculated by the calculation processing.

### REFERENCE SIGNS LIST

10 Control device
11 Input reception section
12 Camera
13 Control unit
14 Display section
15 Storage section
121 Depth camera
122 Color camera
132 Calculation unit
133 Determination unit
134 Model control unit (control unit)
135 Display control unit (control unit)
1311 Recognition processing unit
13111 Hand recognition processing unit
13112 Upper body recognition processing unit
13113 Face recognition processing unit

1. A control device comprising:
an acquisition unit configured to acquire position information on fingers and elbows related to positions of fingers and elbows of a user;
a calculation unit configured to calculate elbow joint orientation information related to orientations of joints of the elbows, based on the position information on the fingers and the elbows acquired by the acquisition unit; and
a control unit configured to control a posture of an avatar of the user, based on the elbow joint orientation information calculated by the calculation unit.

2. The control device according to claim 1, wherein
the calculation unit further calculates finger joint orientation information related to orientations of joints of the fingers, based on the position information on the fingers, and
the control unit controls the posture of the avatar, based additionally on the finger joint orientation information calculated by the calculation unit.

3. The control device according to claim 1, wherein
the acquisition unit acquires the position information on the fingers and the elbows, based on depth information on the fingers and the elbows.

4. The control device according to claim 1, wherein
the acquisition unit further acquires position information on at least one of hands, shoulders, elbows, a head, and a neck related to a position of at least one of hands, shoulders, elbows, a head, a face, and a neck of the user, and
the calculation unit calculates the elbow joint orientation information, based additionally on the position information on at least one of the hands, the shoulders, the elbows, the head, and the neck acquired by the acquisition unit.

5. The control device according to claim 4, wherein
the acquisition unit acquires the position information on at least one of the hands, the shoulders, the elbows, the head, and the neck, based on a feature point of an upper body of the user in depth information on the upper body.

6. The control device according to claim 4, wherein
the acquisition unit acquires position information on the hands and the elbows, and
the calculation unit calculates the elbow joint orientation information, based on the position information on the hands and the elbows acquired by the acquisition unit.

7. The control device according to claim 6, wherein
the calculation unit calculates the elbow joint orientation information by performing inverse kinematics (IK) processing on the position information on the hands and the elbows acquired by the acquisition unit.

8. The control device according to claim 1, wherein
the acquisition unit further acquires position information on hands, shoulders, a neck, and elbows related to positions of hands, shoulders, a neck, and elbows, and
the calculation unit estimates shoulder joint orientation information related to orientations of joints of the shoulders of the avatar with respect to a direction directly facing a screen displayed on the control device and to a vertical direction, based on the position information on the hands, the shoulders, the neck, and the elbows acquired by the acquisition unit.

9. The control device according to claim 8, wherein
when position information on a face related to a position of a face of the user is not acquired by the acquisition unit, the calculation unit estimates the position information on the face, based on the position information on the hands, the shoulders, the neck, and the elbows acquired by the acquisition unit.

10. The control device according to claim 9, wherein
the acquisition unit further acquires calibration information for calibrating the posture of the avatar, and
when the calibration information is acquired by the acquisition unit, the control unit calibrates the posture of the avatar so that a waist of the avatar is positioned vertically below a head of the avatar corresponding to the position information on the face estimated by the calculation unit.

11. The control device according to claim 10, wherein
the acquisition unit acquires the calibration information when posture information is acquired indicating that the user or the avatar is in a specific posture.

12. The control device according to claim 11, wherein
the acquisition unit acquires the calibration information when the posture information is acquired over a predetermined time period or longer.

13. The control device according to claim 12, wherein
the acquisition unit acquires the calibration information when the posture information is acquired over two seconds or more.

14. The control device according to claim 10, wherein
during calibration by the control unit, the control unit displays a range corresponding to an imaging range of a user on a screen on which the avatar is displayed.

15. The control device according to claim 2, further comprising
a determination unit configured to determine whether the user is in a specific posture, wherein
when the determination unit determines that the user is in the specific posture, the control unit controls the posture of the avatar to be a posture corresponding to the specific posture.

16. The control device according to claim 15, wherein
the acquisition unit further acquires posture information related to the specific posture, and
the determination unit determines whether the user is in the specific posture, based on the posture information, the position information on the fingers and the elbows, the finger joint orientation information, and the elbow joint orientation information, which are acquired by the acquisition unit.

17. The control device according to claim 16, wherein
the acquisition unit acquires, as the posture information related to the specific posture, posture information related to a posture in which the user is playing a musical instrument, a posture in which arms are folded, or a posture in which hands are joined together behind a head, and
when the determination unit makes a determination that the user is in the posture in which the user is playing the musical instrument, the posture in which the arms are folded, or the posture in which the hands are joined together behind the head, the control unit controls the posture of the avatar to be a posture corresponding to the posture for which the determination has been made by the determination unit, among the posture in which the musical instrument is played, the posture in which the arms are folded, and the posture in which the hands are joined together behind the head.

18. The control device according to claim 17, wherein
the acquisition unit further acquires imaging information on the user obtained by imaging the user, and
the determination unit determines whether the user is in the specific posture, based additionally on at least one of a situation represented by the imaging information on the user, a predetermined object included in the imaging information on the user, and input information on the user.

19. A control method executed by a control device, the method comprising:
an acquisition step of acquiring position information on fingers and elbows related to positions of fingers and elbows of a user;
a calculation step of calculating elbow joint orientation information related to orientations of joints of the elbows, based on the position information on the fingers and the elbows acquired in the acquisition step; and
a control step of controlling a posture of an avatar of the user, based on the elbow joint orientation information calculated in the calculation step.

20. A control program that causes a computer mounted on a control device to execute:
acquisition processing of acquiring position information on fingers and elbows related to positions of fingers and elbows of a user;
calculation processing of calculating elbow joint orientation information related to orientations of joints of the elbows, based on the position information on the fingers and the elbows acquired by the acquisition processing; and
control processing of controlling a posture of an avatar of the user, based on the elbow joint orientation information calculated by the calculation processing.
